(19) United States
(12) Patent Application Publication
(10) Pub. No.: US 2021/0163889 A1
(43) Pub. Date: Jun. 3, 2021

(54) PREMATURE INFANT SKIN MODEL AND METHOD OF CREATING THE SAME

(71) Applicant: KIMBERLY-CLARK WORLDWIDE, INC., Neenah, WI (US)

(72) Inventors: Penkanok Sriwiriyanont, Neenah, WI (US); Debbie Y. Ngai, Neenah, WI (US); Elizabeth S. Weir, Neenah, WI (US); Karien Rodriguez, Roswell, GA (US)

(73) Assignee: KIMBERLY-CLARK WORLDWIDE, INC., Neenah, WI (US)

(21) Appl. No.: 16/649,877

(22) PCT Filed: Sep. 25, 2018
(86) PCT No.: PCT/US18/52520

§ 371 Date: Mar. 23, 2020

Related U.S. Application Data
(60) Provisional application No. 62/563,216, filed on Sep. 26, 2017.

(51) Int. Cl. C12N 5/071; C12N 5/00; G01N 33/50

(52) U.S. Cl. CPC C12N 5/0625; C12N 5/0062; G01N 33/5088; C12N 2503/06; C12N 2513/00; C12N 2501/2306; C12N 2503/02; G01N 33/5044

(57) ABSTRACT

A premature infant skin model and methods of creating the same are disclosed. One method of creating a three-dimensional premature infant skin model can include providing neonatal skin cells, exposing the neonatal skin cells to a treatment solution including interleukin-6 for a treatment period; and removing the treatment solution from the neonatal skin cells after the treatment period to provide the three-dimensional premature infant skin model.

### TECHNICAL FIELD

The present disclosure relates to a premature infant skin model and methods for creating the same. More specifically, the present disclosure relates to a three-dimensional premature infant skin model and methods for creating the same.

### BACKGROUND OF THE DISCLOSURE

According to Centers for Disease Control and Prevention (CDC), in 2014, 1 in 9 or approximately 450,000 babies were born prematurely (i.e., born at less than 37 weeks of gestational age) in the United States alone. While skin is the human body's major barrier to external insults, premature skin lacks some characteristics of mature skin that can serve as a barrier. Due to the insufficient time in utero, the skin of premature infants is not completely developed at birth. Specifically, premature skin lacks a fully-developed stratum corneum, which plays crucial roles in preventing infection from external stimuli and in the regulation of body temperature. As a result, premature skin is more susceptible to irritation from external stimuli, such as urine and fecal matter, and more predisposed to infection. Dermal instability is another complication in premature infant skin. Insufficient collagens and elastic fibers not only may cause edema, but can also make the skin more susceptible to pressure and ischemic injury. Furthermore, immature dermo-epidermal junction can result in damage when adhesive materials are applied in Neonatal Intensive Care Unit (NICU) settings.

Primary skin cells or explants from human subjects have been used in in-vitro studies for skin research. Reconstructed three-dimensional skin models have also been prepared to study skin and products interacting with the skin. Such reconstructed skin models can be useful tools for in-vitro bench testing in active screening and conducting research on a variety of products and formulations, and can provide helpful information prior to any clinical testing. However, reconstructed skin models have focused on mature skin, or even aged skin, and thus, are not accurate representations of how a premature infant's skin reacts to external stimuli and/or develops.

Thus, there is a desire for a premature infant skin model that can be used to further understand premature infant skin behavior. There is also a desire for a method to create a premature infant skin model that is consistent and replicates premature infant skin.

### SUMMARY OF THE DISCLOSURE

In one aspect of the disclosure, a method of creating a premature infant skin model is provided. The method can include providing neonatal skin cells. The method can further include exposing the neonatal skin cells to a treatment solution comprising interleukin-6 for a treatment period. The method can also include removing the treatment solution from the neonatal skin cells after the treatment period to provide the three-dimensional premature infant skin model.

In another aspect of the disclosure, a method of creating a three-dimensional premature infant skin model is provided that includes providing neonatal skin cells that include neonatal dermal skin cells and neonatal epidermal skin cells. The method can also include exposing the neonatal skin cells to a treatment solution comprising interleukin-6 for a treatment period. The interleukin-6 can be provided at a concentration of from about 10 ng/mL to about 100 ng/mL by total weight/volume of the treatment solution. The method can additionally include removing the treatment solution from the neonatal skin cells after the treatment period to provide the three-dimensional premature infant skin model.

### DESCRIPTION OF THE DRAWINGS

FIG. 1 illustrates a diagram of one embodiment of a method to provide a three-dimensional premature infant skin model.

FIG. 2 illustrates Transepithelial Electrical Resistance (TEER) readings for various untreated samples of fully-developed skin equivalents, highlighting significant variance in initial TEER readings for samples across plates.

FIG. 3 illustrates initial TEER readings being used to randomize samples from FIG. 2 across treatment codes before experimentation to reduce between-plate variance.

FIG. 4A illustrates IL-6 readings from fully-developed skin models and under-developed skin models exposed to urine simulant for 24 hours.

FIG. 4B illustrates IL-8 readings from fully-developed skin models and under-developed skin models exposed to urine simulant for 24 hours.

FIG. 4C illustrates IL-1a readings from fully-developed skin models and under-developed skin models exposed to urine simulant for 24 hours.

FIG. 4D illustrates TEER readings from fully-developed skin models and under-developed skin models exposed to urine simulant for 24 hours.

FIG. 5A illustrates a Hematoxylin and Eosin (H&E) stained histology image of an under-developed comparative skin model not exposed to a treatment solution including IL-6.

FIG. 5B illustrates an H&E stained histology image of an under-developed skin model exposed to a treatment solution including IL-6.

FIG. 6 illustrates TEER readings for an under-developed comparative skin model not exposed to a treatment solution including IL-6 and an under-developed skin model exposed to a treatment solution including IL-6 over various days in a treatment period.

FIG. 7A illustrates proliferating nuclei antigen (Ki67) immunostaining in a photomicrograph of an under-developed comparative skin model not exposed to a treatment solution including IL-6.

FIG. 7B illustrates proliferating nuclei antigen (Ki67) immunostaining in a photomicrograph of an under-developed skin model exposed to a treatment solution including IL-6.

FIG. 8 illustrates Ki67 positive (Ki67+) cells in an under-developed comparative skin model not exposed to a treatment solution including IL-6 and an under-developed skin model exposed to a treatment solution including IL-6 over various days in a treatment period, with the number of Ki67+nuclei in each photomicrograph (FIG. 7A and 7B, respectively) being analyzed using the IHC Tool plugin in ImageJ and results were plotted as mean±se.

FIG. 9A illustrates the overlap of differentially expressed (DE) genes due to IL-6 treatment on each experimental day (1, 3, and 7) identified by microarray.

FIG. 9B illustrates a Principle Component Analysis (PCA) using the selected 3365 DE genes from FIG. 9A.

FIG. 9C illustrates a hierarchical clustering of the selected 3365 DE genes from FIG. 9A.

FIG. 10 illustrates a bar graph of biological pathways that are related to IL-6 treatment over time with the ratio of listed genes found in each pathway over the total number of genes in that particular pathway.

FIG. 11 illustrates TEER and skin capacitance readings (DPM) of a fully-developed skin model and an under-developed skin model not exposed to a treatment solution including IL-6 that were each exposed to phosphate buffered saline (PBS) serving as a control, urine simulant, fecal irritant, and urine simulant/fecal irritant combination.

FIG. 12 illustrates TEER and skin capacitance readings (DPM) of an under-developed comparative skin model not exposed to a treatment solution including IL-6, and an under-developed skin model exposed to a treatment solution including IL-6 that were each exposed to phosphate buffered saline (PBS) serving as a control, urine simulant, fecal irritant, and urine simulant/fecal irritant combination.

FIGS. 13A-13C illustrate H&E stained histology images of a fully-developed skin model, an under-developed comparative skin model not exposed to a treatment solution including IL-6, and an under-developed skin model exposed to a treatment solution including IL-6, respectively, each exposed to PBS.

FIGS. 14A-14C illustrate H&E stained histology images of a fully-developed skin model, an under-developed comparative skin model not exposed to a treatment solution including IL-6, and an under-developed skin model exposed to a treatment solution including IL-6, respectively, each exposed to urine simulant.

FIG. 15 illustrates an H&E stained histology image of a fully-developed skin model exposed to fecal irritant.

FIGS. 16A-16C illustrate H&E stained histology images of a fully-developed skin model, an under-developed comparative skin model not exposed to a treatment solution including IL-6, and an under-developed skin model exposed to a treatment solution including IL-6, respectively, each exposed to urine simulant/fecal irritant combination.

FIG. 17 illustrates skin capacitance readings (DPM) for an under-developed skin model exposed to a treatment solution including 10 ng/mL of IL-6 for a treatment period of three days and then exposed to PBS or a urine simulant/fecal irritant combination for periods of 10, 30, and 60 minutes.

FIG. 18A illustrates H&E stained histology image of an under-developed skin model exposed to a treatment solution including 10 ng/mL of IL-6 for a treatment period of three days and then exposed to PBS for 10 minutes.

FIGS. 18B-D illustrate H&E stained histology image of an under-developed skin model exposed to a treatment solution including 10 ng/mL of IL-6 for a treatment period of three days and then exposed to a urine simulant/fecal irritant combination for 10, 30, and 60 minutes, respectively.

FIG. 19 illustrates skin capacitance readings (DPM) of an under-developed comparative skin model not exposed to a treatment solution including IL-6 and an under-developed skin model exposed to a treatment solution including IL-6, each exposed to PBS and a urine simulant/fecal irritant combination for 10 minutes and 30 minutes.

FIG. 20A illustrates multiplex immunoassay testing results for GM-CSF for an under-developed comparative skin model not exposed to a treatment solution including IL-6 and an under-developed skin model exposed to a treatment solution including IL-6, each exposed to PBS (control) and a urine simulant/fecal irritant combination for 30 minutes.

FIG. 20B illustrates multiplex immunoassay testing results for IL-1a for an under-developed comparative skin model not exposed to a treatment solution including IL-6 and an under-developed skin model exposed to a treatment solution including IL-6, each exposed to PBS (control) and a urine simulant/fecal irritant combination for 30 minutes.

FIG. 20C illustrates multiplex immunoassay testing results for IL-8 for an under-developed comparative skin model not exposed to a treatment solution including IL-6 and an under-developed skin model exposed to a treatment solution including IL-6, each exposed to PBS (control) and a urine simulant/fecal irritant combination for 30 minutes.

FIG. 20D illustrates multiplex immunoassay testing results for IL-10 for an under-developed comparative skin model not exposed to a treatment solution including IL-6 and an under-developed skin model exposed to a treatment solution including IL-6, each exposed to PBS (control) and a urine simulant/fecal irritant combination for 30 minutes.

FIG. 20E illustrates multiplex immunoassay testing results for MCP-1 for an under-developed comparative skin model not exposed to a treatment solution including IL-6 and an under-developed skin model exposed to a treatment solution including IL-6, each exposed to PBS (control) and a urine simulant/fecal irritant combination for 30 minutes.

FIG. 21 illustrates multiplex immunoassay testing results for IL-1a, GM-CSF, and MCP-1 for an under-developed skin model not exposed to a treatment solution including IL-6 and an under-developed skin model exposed to a treatment solution including IL-6, each exposed to PBS (control) and a urine simulant/fecal irritant combination for exposure times of 10 minutes and 30 minutes.

FIG. 22 illustrates protein analysis results for an under-developed skin model not exposed to a treatment solution including IL-6 and an under-developed skin model exposed to a treatment solution including IL-6, each exposed to a urine simulant/fecal irritant combination for 30 minutes.

FIG. 23 illustrates a schematic diagram of an RNA microarray sample analysis as described in the Procedures section herein.

### DETAILED DESCRIPTION OF THE DISCLOSURE

The present disclosure generally relates to a premature infant skin model that includes structural and molecular changes to skin equivalents (i.e., not human skin) that resemble features found in premature infant skin.

A commercially available three-dimensional skin model was used as the subject of treatment in producing a three-dimensional premature infant skin model 10. The three-dimensional skin model that was used in the experiments discussed herein was a skin equivalent produced by MatTek Corporation (Ashland, Mass.), model number EFT-400-7A that was derived from neonatal foreskin. The skin equivalent included neonatal dermal skin cells and neonatal epidermal skin cells. However, it is believed that the techniques and procedures discussed herein would be as effective at producing a physiologically relevant three-dimensional premature infant skin model 10 with other available three-dimensional skin equivalents. It is also believed that premature infant skin models 10 could be developed and useful for various purposes that include only neonatal epidermal skin cells.

Several samples of skin equivalents were transferred to six-well plates each containing 2.5 mL of a treatment solution. As shown in Table 1, a skin equivalent model including neonatal dermal skin cells and neonatal epidermal skin cells, model number EFT-400-7A, as referenced above was used for samples 1A, 1B, 2A, and 2B in these experiments. Samples 1A, 1B, 2A, and 2B were put in culture mediums B, C, and D (available from MatTek Corporation) according to the schedule as shown in Table 1. Culture Mediums B, C, and D include: the base medium of Dulbecco's Modified Eagle Medium (DMEM), growth factors/hormones of: epidermal growth factor (EGF), insulin, hydrocortisone, and other stimulators, an antibiotic of gentamicin of 5 μg/mL, an antifungal agent of amphotericin B of 0.25 μg/mL, a pH indicator of phenol red, and other lipid precursors, such as linoleic acid, used to enhance epidermal barrier formation. Of course, it is expected that various other skin growth culture mediums could be used. For example, EpiLife keratinocyte culture medium (Thermo Fisher Scientific) supplemented with HKGS kit (Thermo Fisher Scientific) can be used in the airlifting procedure by adjusting calcium level and adding growth and/or differentiation promoting agents as well as lipid precursors, such as progesterone, ascorbic acid and linoleic acids. Alternatively, CnT-Prime Airlift (Cell-n-Tec) can be utilized for the same purpose. In the experiments, these culture mediums B, C, and D were then each supplemented with 10 ng/mL of interleukin-6 (“IL-6”) to provide treatment solutions referred to as B*, C*, D* used for sample 1B, and B* and C* used for sample 2B as noted in Table 1. A fully developed skin equivalent model, produced by MatTek Corporation (Ashland, Mass.), model number EFT-400, was used as a comparative sample, and is referred to herein as sample 3. The fully developed skin equivalent model (sample 3), is the same as the sample 1A after day 7, and then is placed in a DMEM based assay medium (referenced as “ASY”), available from MatTek Corporation. As will be described further below, an “X” in Table 1 indicates the day in which an insult of exudate was added to the top of the skin equivalent and then washed off after the insult time, as will be discussed further below.

TABLE 1
Experimental schedule for treatment solution changes and insults.

| Sample | | | | | | | | | |
|---|---|---|---|---|---|---|---|---|---|
| No. | Sample Name | Day 0 | Day 1 | Day 2 | Day 3 | Day 4 | Day 5 | Day 6 | Day 7 |
| 1A | Skin Development | B | B | C | C | C | C | D | D |
| | (no IL-6) | | | | | | | | |
| 1B | Skin Development | B* | B* | C* | C* | C* | C* | D* | D* |
| | (with IL-6) | | | | | | | | |
| 2A | Under Developed | B | B | C | C, X | | | | |
| | Skin (no IL-6) | | | | | | | | |
| 2B | Under Developed | B* | B* | C* | C*, X | | | | |
| | Skin (with IL-6) | | | | | | | | |
| 3 | Fully Developed | ASY | X | | | | | | |
| | Skin (no IL-6) | | | | | | | | |

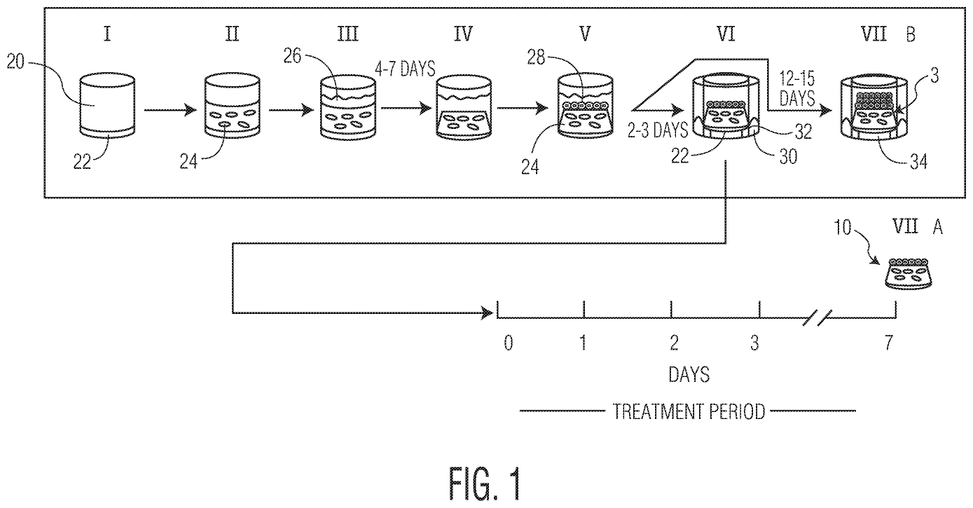

In the present experimentation, the neonatal skin cells were provided via a commercial skin equivalent as noted above, and included both neonatal dermal skin cells 24 and neonatal epidermal skin cells 28. As illustrated in FIG. 1, a method of providing a premature infant skin model 10 can include providing these neonatal skin cells 24, 28 from a skin equivalent (such as shown in step VI). These neonatal skin cells 24, 28 were then exposed to a treatment solution 30 including IL-6 for a treatment period, and then the treatment solution 30 was removed from the neonatal skin cells 24, 28 to provide a three-dimensional premature infant skin model 10.

However, as illustrated in FIG. 1, it is contemplated that the neonatal dermal skin cells 24 can be provided in a container 20 with a microporous membrane 22 (steps I and II). The method can include culturing the neonatal dermal skin cells 24 in a culture medium 26 for an initial culture period (step III). During the initial culture period, contraction can occur (step IV). In some embodiments, the initial culture period can range between four to seven days. The method can also include adding neonatal epidermal skin cells 28 on top of the neonatal dermal skin cells 24 after the initial culture period for a secondary culture period (step V). In some embodiments, the secondary culture period can range between two to three days. The co-cultured neonatal skin cells 24, 28 and microporous membrane 22 are then lifted to the air-liquid interface 32 after the secondary culture period (step VI). The method can then include exposing the neonatal skin cells 24, 28 to a treatment solution 30 including IL-6 for a treatment period after the secondary culture period, as shown in FIG. 1, and as mentioned above (step VII A). As will be discussed in more detail below, the treatment period can range from one to seven days. In preferred embodiments, the treatment period can range from two days to five days. In a particularly preferred embodiment, the treatment period can be three days.

If after the secondary culture period and culturing of the epidermal skin cells 28 the neonatal skin cells 24, 28 were further cultured without culture medium 34 that did not include IL-6, then the skin model could be developed as a fully developed skin model 36 (step VII B), as shown in FIG. 1. The further culturing period could be for a period of twelve to fifteen days.

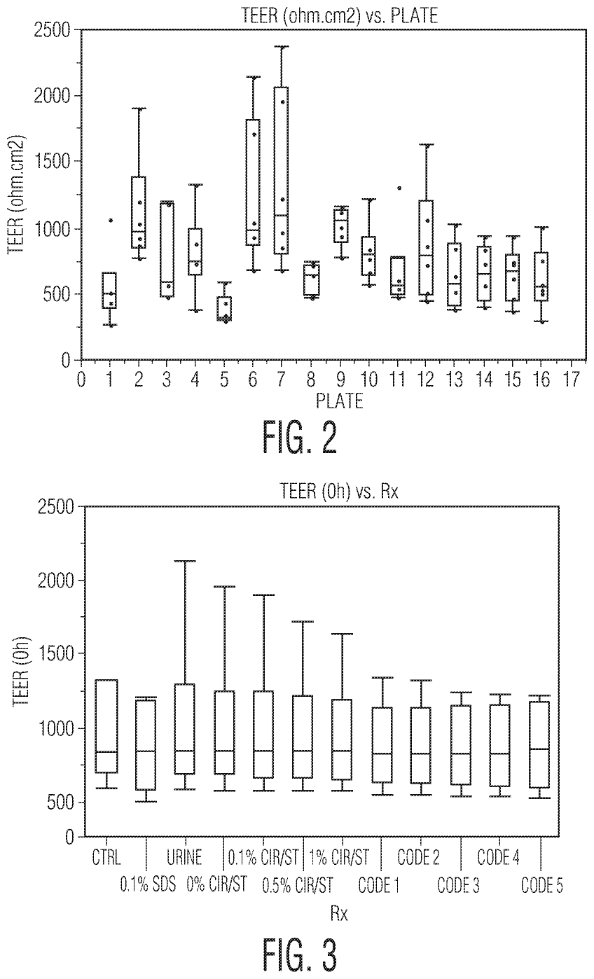

Several samples of skin equivalents were tested for sample-to-sample variability. Through Transepithelial Electrical Resistance (“TEER”) testing, which is described in further detail in the Procedures section herein, it was determined that high variability existed between samples. To minimize the experimental bias, the skin equivalents that were exposed to a treatment solution and the skin equivalents that were being used as a control were assigned based on the results from this non-destructive biophysical measurement. Examples of group assignment based TEER testing are shown in FIGS. 2 and 3.

As illustrated in FIG. 2, a high amount of variability of initial TEER testing values are shown between samples within and between each culture plate. FIG. 3 demonstrates an example of how TEER testing values were used to randomize samples before an experiment. To do this, each sample is initially assigned a number on each plate. Next, the TEER value is measured for each sample. A table with the TEER values are then created and sorted from highest to lowest. Across plates/treatments, samples are then re-assigned, such that the overall average TEER per plate/treatment is about the same. Samples are then moved accordingly to different plates once re-assignment is complete. This procedures provides a more even distribution and reduced variability amongst treatment codes that is shown in FIG. 3.

To study the characteristics of the skin models 10 exposed to treatment solutions of IL-6 and comparative skin models not exposed to treatment of IL-6, a pilot study was conducted to evaluate urine simulant on the skin models. This was accomplished by placing an 8 mm punch of absorbent paper soaked with urine simulant on top of the skin equivalent, as noted in the schedule of Table 1. The urine simulant was prepared according to the formulation in the Procedures section herein. After 24 hours, the paper was removed and the skin equivalent surface was washed three times in 200 μL of sterile phosphate buffered saline (PBS). Biophysical measurements and sample collection were carried out for further analysis, as will be discussed further below.

In subsequent studies, fecal irritants were also tested on these skin models. Severe barrier damage was observed, especially in the under-developed skin models. Therefore, the exposure/collection protocol was modified in order to compare effects of these irritants on different types of the skin models.

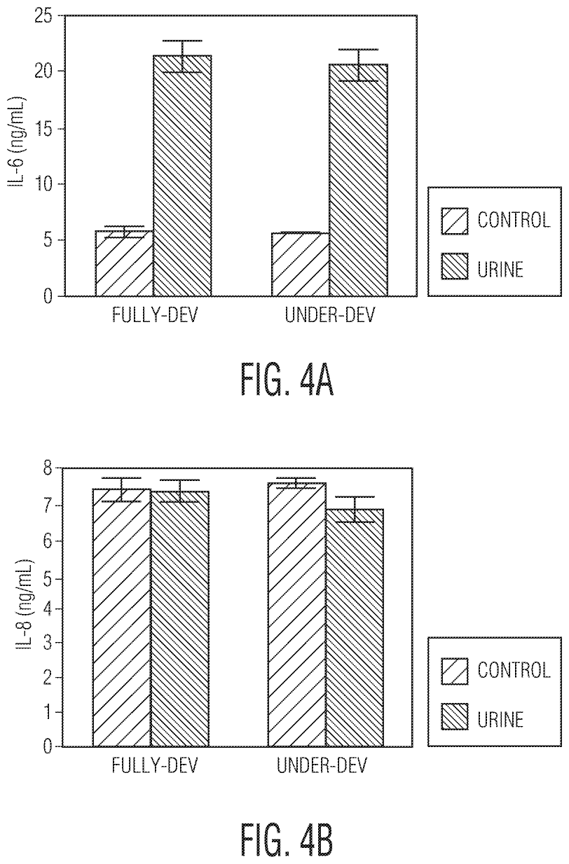

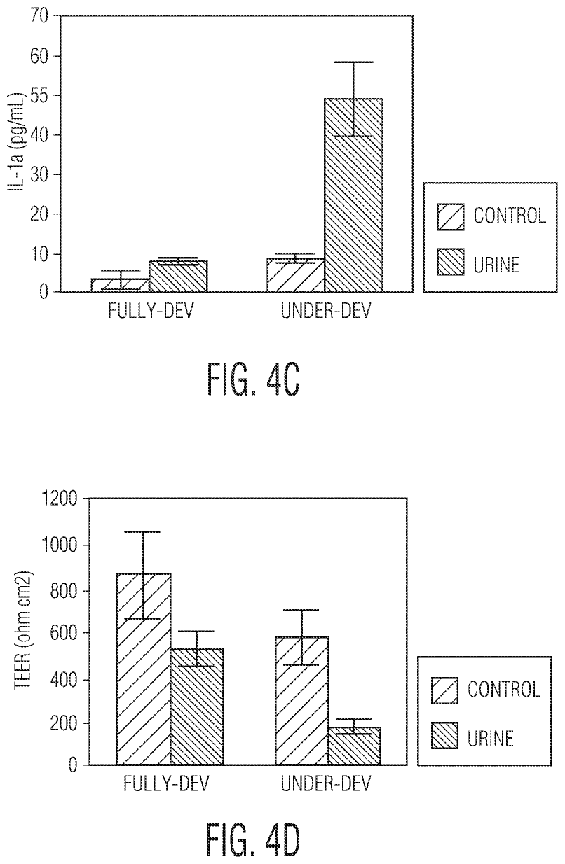

In order to understand effects of urine exposure on skin health, fully-developed skin models (e.g., Sample No. 3) and under-developed skin models that were not treated with IL-6 (e.g., Sample No. 2A) were treated with urine simulant as described in the Procedures section herein. Immunoassay testing for IL-1a, IL-6 and IL-8 (described in the Procedures section herein), as well as TEER testing was conducted. As illustrated in FIGS. 4A-4D, decreased TEER values and increased IL-1a were observed after 24 hours of urine exposure, especially in the under-developed skin models, which lacked a fully-developed stratum corneum (see FIG. 4C). Interestingly, while there was no difference in IL-8 levels between fully-developed and under-developed skin models, a significant increase in IL-6 was detected following urine exposure with no discernible differences between fully-developed and under-developed skin models (see FIG. 4A). Elevation of IL-6 following urine exposure in both skin models suggested that this phenomenon was not barrier dependent. As a result, urine simulant testing induced an innate immune response and deteriorated skin barrier health of both fully-developed skin models (Sample No. 3) as well as the under-developed skin models (Sample No. 2A). From this testing, it was determined that further testing on IL-6 should be studied to mimic the in utero environment of premature infants.

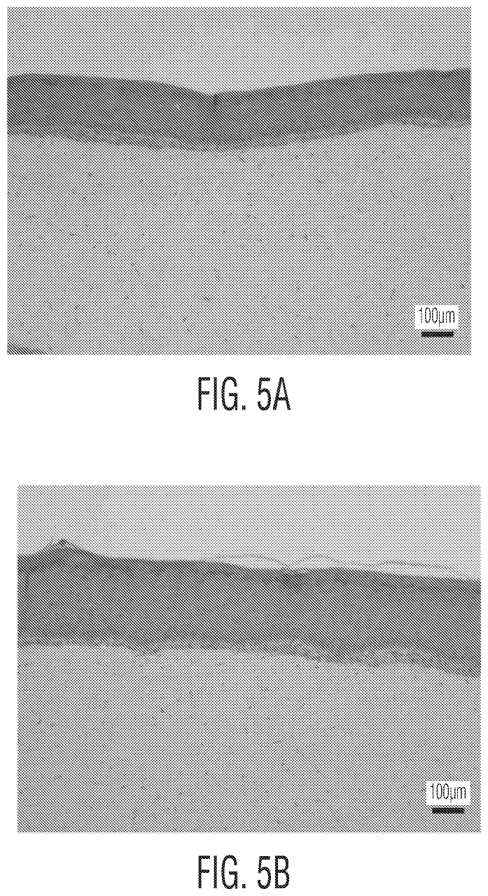

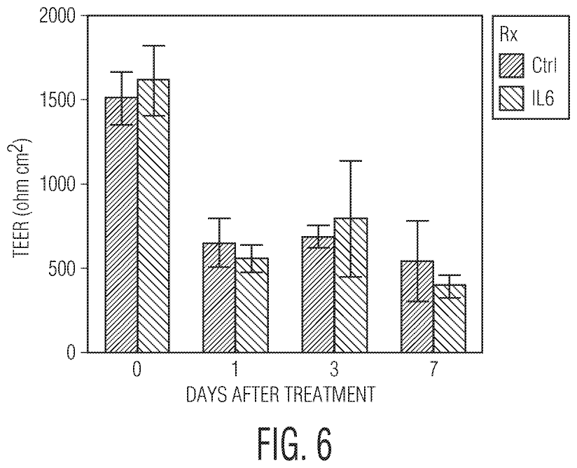

To investigate the effect of IL-6 during skin development, evaluation of the concentration of IL-6 in the treatment solution 30 was tested at 10, 50, and 100 ng/mL. After conducting TEER testing, the TEER values showed no difference among skin models 10 treated with the IL-6 (e.g., sample nos. 1B and 2B) and the comparative or control samples that included a culture medium without any IL-6 (e.g., samples 1A and 2A). However, histological sections of the skin models 10 exposed to IL-6 clearly showed effects of IL-6 even in the cultured skin group that received the lowest IL-6 dosage of 10 ng/mL. An experiment was then repeated using only 0 and 10 ng/mL of IL-6. Samples were collected on day 0, 1, 3, and 7. Similar to the results from a pilot run, epidermal hyperplasia was detected in skin models 10 exposed to a treatment solution 30 including IL-6 compared to skin samples that were not exposed to a treatment solution 30 including IL-6. FIG. 5A provides a representation of a histological image of a comparative skin sample that was not exposed to a treatment solution 30 including IL-6 after 3 days (sample no. 2A from Table 1). FIG. 5B provides a representative sample of a histological image of a skin model 10 that was exposed to a treatment solution 30 including IL-6 at 10 ng/mL after 3 days (sample no. 2B from Table 1). Again, there were no differences in TEER readings for the skin model 10 exposed to a treatment solution 30 compared to control skin samples not exposed to the treatment solution 30 including IL-6 (FIG. 6), regardless of the period of time that has passed in the treatment period.

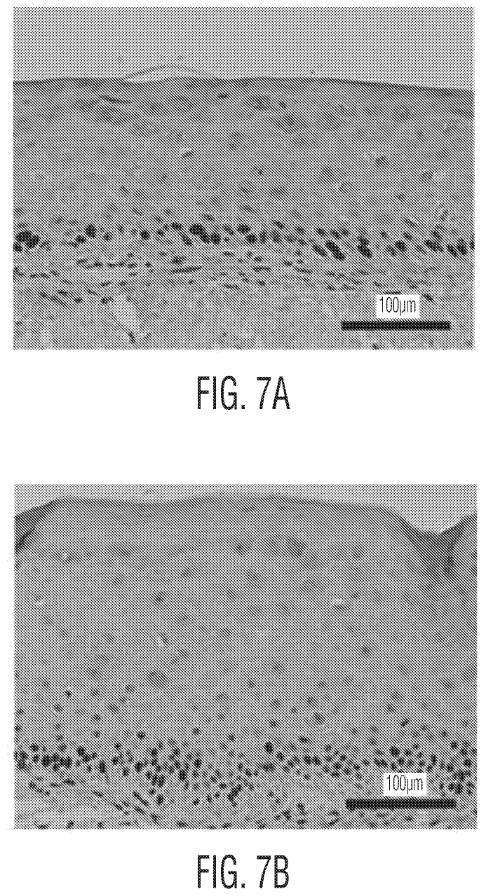

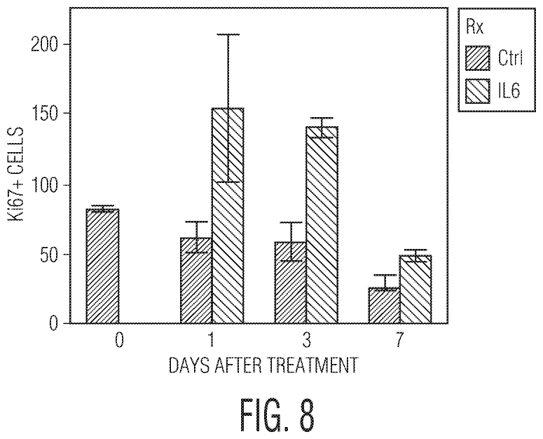

FIG. 7A illustrates a comparative skin sample at day 3 that was not treated with a treatment solution 30 including IL-6 and FIG. 7B illustrates a skin model 10 that was treated with a treatment solution 30 including IL-6 for a treatment period of 3 days. The Ki67+nuclei are shown in a dark brown stained color from 3,3′-diaminobenzidine substrate that reacts with horseradish peroxidase conjugated antibody in each image. It is important to note that the low dose IL-6 treatment (10 ng/mL) affected nuclei size and increased number of Ki67+cells beyond basal lamina, because Ki67+proliferating cells are typically only observed in the basal layer. Additionally, the nuclei were much smaller as the cells were prompted to develop faster. Thus, exposing neonatal skin cells to a treatment solution 30 including IL-6 induced epidermal hyperplasia (increased Ki67+) without disturbing biophysical properties of a tight junction barrier (no change in TEER). As illustrated in FIG. 8, a significant increase in Ki67+cells was found in skin models 10 treated with a treatment solution 30 including IL-6 as early as a 1 day treatment period, remained high after a 3 day treatment period, and subsided after a 7 day treatment period after the barrier became more completely developed. In FIG. 8, the number of Ki67+nuclei in each were counted from photomicrographs (see FIGS. 7A and 7B) using the IHC Tool Plugin in ImageJ as described in the Procedures section herein and the results were illustrated as mean±se.

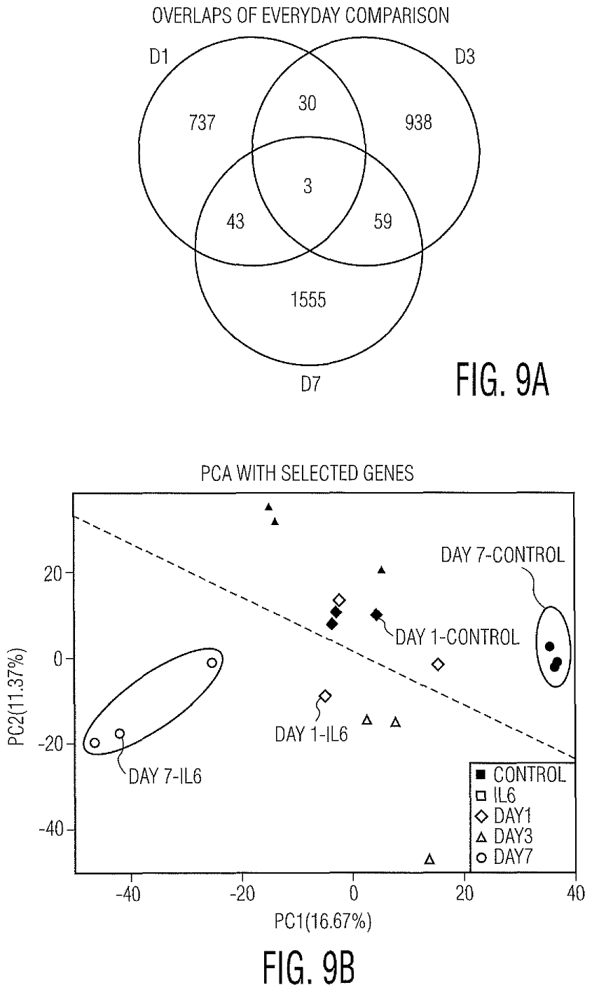

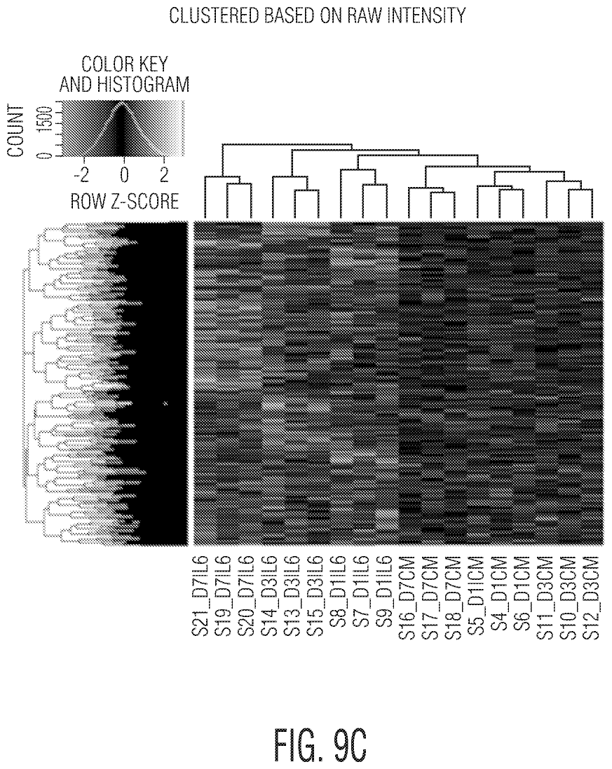

To further investigate effects of IL-6 used in a treatment solution 30 during skin development, microarray analysis was conducted on total RNA prepared from the skin models 10 exposed to IL-6 treatment and comparative models that did not receive the IL-6 treatment. Biopsies were harvested throughout the treatment period. 813 genes were found differentially expressed (DE) due to IL-6 treatment on Day 1; 1030 genes were found differentially expressed due to IL-6 treatment on Day 3; and 1660 genes were found differentially expressed due to IL-6 treatment on Day 7 (See FIG. 9A). Out of the total 3365 differentially expressed genes, 3 were found in common of all three experimental days (FIG. 9A). However, these are non-coding transcripts. Principle Component Analysis (PCA) was run using the selected 3365 DE genes and showed a clear separation of different treatment groups, as shown in FIG. 9B. Dividing the samples that were treated with IL-6 from controls was possible as shown by the dashed line in FIG. 9B. Treatment groups from Day 7 were the most separated as they are located at the opposite ends of the graph showed in circles in FIG. 9B. As illustrated in FIG. 9C, hierarchical clustering of the data with only the 3365 DE genes showed that treated samples were completely separated from control, and that each cluster only contained samples collected within the same experimental day. Gene expression patterns can also be identified from the heatmap of the hierarchical clustering, with the samples being exposed to a treatment solution 30 including IL-6 being on the left half of FIG. 9C and the samples that were not exposed to treatment on the right half of FIG. 9C. In FIG. 9C, the coding on the bottom of the figure includes the sample number, whether the sample was exposed to IL-6 treatment solution, and the number of days (e.g., S21_D7IL6 refers to a sample treated with IL-6 and on Day 7). Day 7 showed the most variance of gene expression.

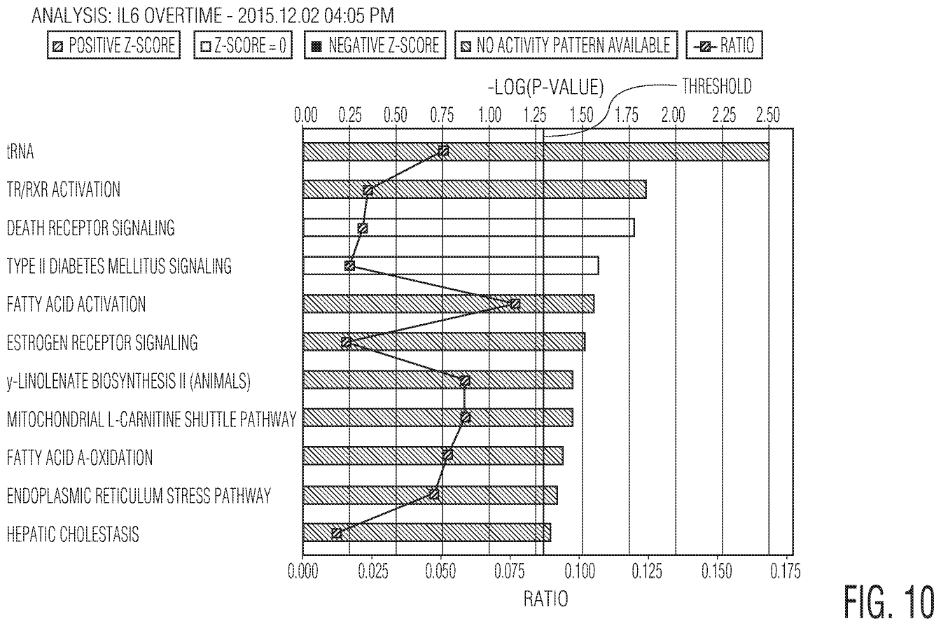

To further analyze DE genes, Ingenuity Pathway Analysis (IPA) software was used to identify top biological networks by ranking them according to a statistical likelihood approach (n=532; enrichment score are represented as -log(P-value), with a threshold of 1.3 as the cut-off of statistical significant (P<0.05)). Interestingly, biological pathways that may be regulated by IL-6 treatment are related to tRNA, retinoid X receptor RXR, estrogen receptor signaling and lipid synthesis (see FIG. 10), as shown by bar graphs with the ratio of listed genes found in each pathway over the total number of genes in that particular pathway. Dysregulation of retinoid X receptor RXR has been previously reported to be associated with preterm labor, whereas estrogen, progesterone and lipid synthesis are known to be involved in the development of epidermal barrier during late gestation.

Multiple Reaction Monitoring, as discussed in the Procedures section herein, was undertaken to study a variety of selected skin barrier proteins in the skin models and comparative skin samples. Changes of skin barrier proteins were evaluated during the treatment period day 1, 3, and 7 for skin models 10 being treated with a treatment solution 30 including IL-6 and for the comparative skin samples not exposed to IL-6. On day one, there was no detectable change of skin barrier proteins in the skin models 10 exposed to IL-6 or the comparative skin samples. However, differences between skin models 10 being exposed to IL-6 and the comparative skin samples not exposed to IL-6 were detected on day three. In particular, six proteins including S100A14, S100A18, S100A7, kallikrein-6, SPINKS, and caspase-14 were found to be expressed at significantly lower levels in the skin models 10 being treated with IL-6. After the skin models 10 become more completely developed on day seven, only one protein, MT-CO2, which is a part of complex IV in the mitochondrial membrane, was expressed at significantly lower level in the skin model 10 that was maturated under IL-6 influence. In all testing, the skin models 10 being exposed to IL-6 treatment solution 30 exhibited reduced levels of S100 proteins compared to the control skin samples not exposed to IL-6.

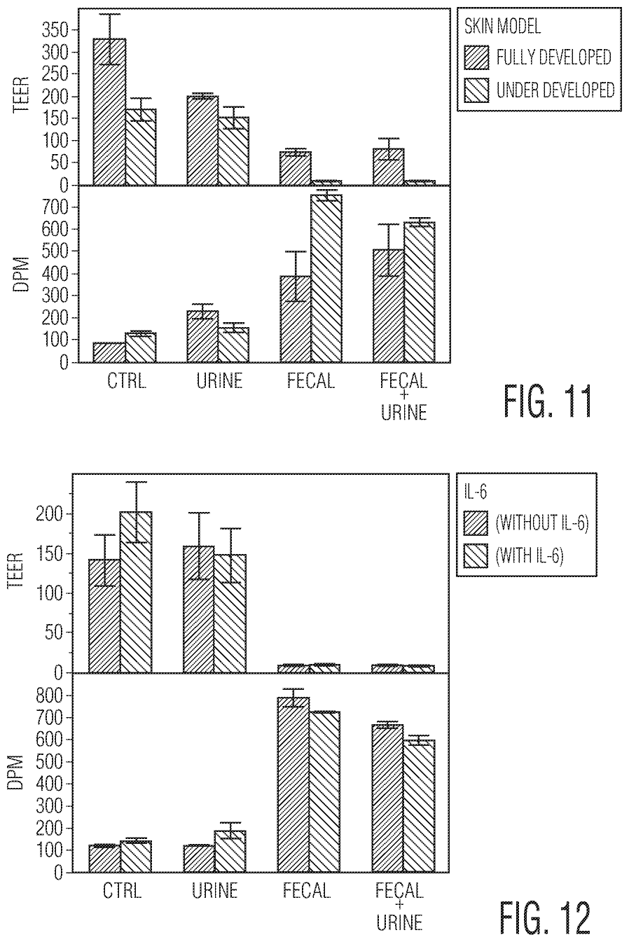

A comparative study was conducted between TEER testing to skin capacitance measured by the use of the NOVA meter (as also described in the Procedures section herein). The skin capacitance testing reports the readout in Dermal Phase Meter (DPM) units. Two skin models, a fully-developed model (sample no. 3 from Table 1, which is the under-developed skin model sample no. 1A from Table 1 after Day 7 and then placed in the DMEM based assay medium, “ASY”) and an under-developed skin model (sample no. 2A from Table 1), neither of which were exposed to IL-6, were exposed to PBS control, urine simulant, fecal irritants, or urine simulant/fecal irritant combination at 1:1 mixture. The fully-developed model was exposed to such simulants/irritants for four hours and the under-developed skin model was exposed to such simulants/irritants for 60 minutes. All tested skin samples were washed, dabbed dry, and allowed to recover in the incubator at 37° C./5% CO₂ overnight before TEER testing and capacitance measurements were taken. Based on visual observation, exposure to fecal irritant or urine simulant/fecal irritant combination caused serious damage to the skin samples. Phenol red containing liquid was observed on the skin surface. Corresponding to visual observation, skin samples treated with fecal irritant only or fecal irritant mixed with urine simulant had very low TEER testing measurements and extremely high DPM readouts, which is illustrated in FIG. 11.

A comparison of TEER testing measurements to skin capacitance readings was undertaken of the under-developed skin models 10 exposed to IL-6 and under-developed skin models not exposed to IL-6 when both models were exposed to PBS, urine simulant, fecal irritant, and a combination of urine simulant and fecal irritant for 60 minutes. The results of this testing are illustrated in FIG. 12. No statistical difference was exhibited between the under-developed skin model 10 being exposed to IL-6 from the under-developed skin model not exposed to IL-6 when exposed to such simulants/irritants for sixty minutes. However, varying the simulant/irritant exposure time, as discussed later herein, did provide a difference between models.

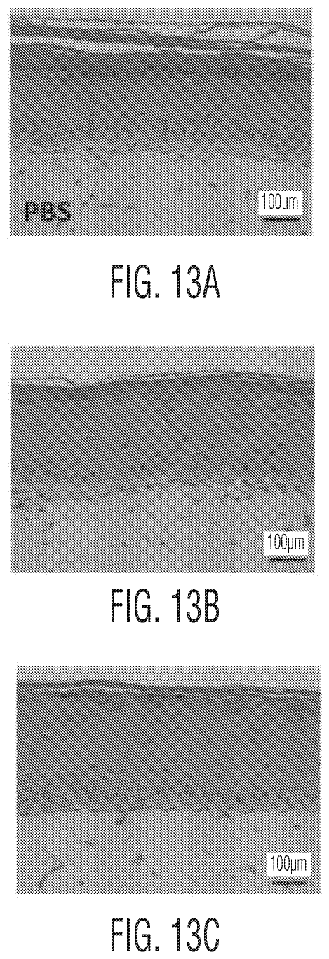

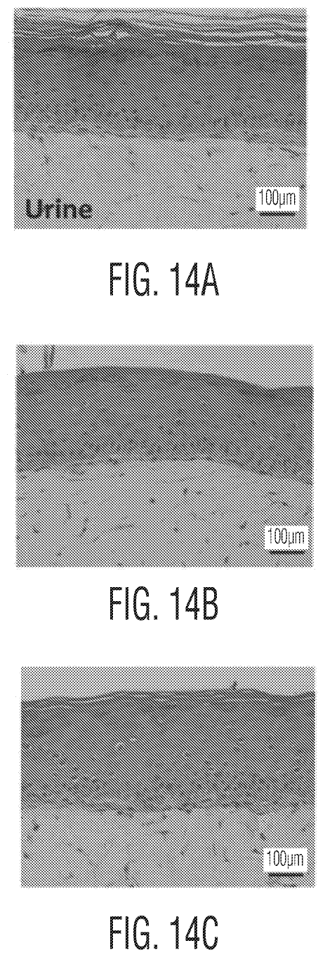

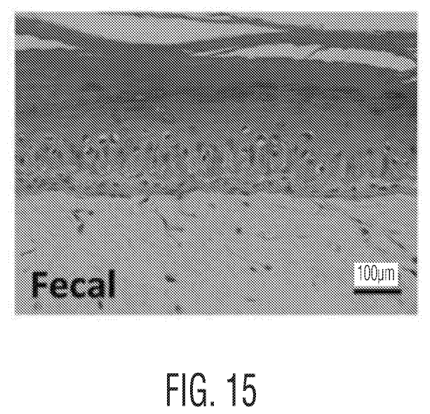

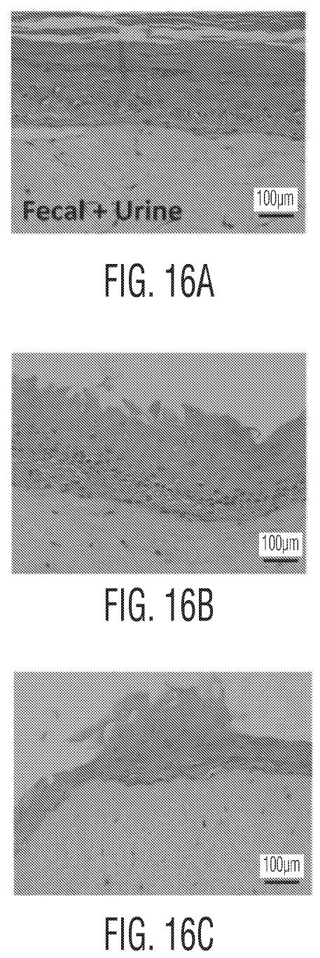

Hematoxylin and Eosin (H&E) staining also supported the findings of damage to the skin, as illustrated in FIGS. 13A-16C. All of the under-developed skin samples from FIGS. 13A-16C were taken from skin samples after day 3, whereas the fully-developed skin samples (sample no. 3 from Table 1) were taken after the samples had been in the DMEM based assay medium (ASY) for one day (again, the fully-developed skin sample no. 3 is the same as the under-developed skin sample no. 1A from table 1 after Day 7). FIGS. 13A-13C illustrate histological images of a fully-developed skin model, an under-developed comparative skin model not exposed to IL-6, and an under-developed skin model 10 exposed to IL-6, respectively, after being exposed to PBS. FIGS. 14A-14C illustrate histological images of a fully-developed skin model, an under-developed comparative skin model not exposed to IL-6, and an under-developed skin model 10 exposed to IL-6, respectively, after being exposed to urine simulant. FIG. 15 illustrates a histological image of a fully developed skin model after being exposed to fecal irritant (no histological images were taken for the under-developed skin models exposed to fecal irritant due to severe damage). FIGS. 16A-16C illustrate histological images of a fully-developed skin model, an under-developed comparative skin model not exposed to IL-6, and an under-developed skin model 10 exposed to IL-6, respectively, after being exposed to urine simulant and fecal irritant combination.

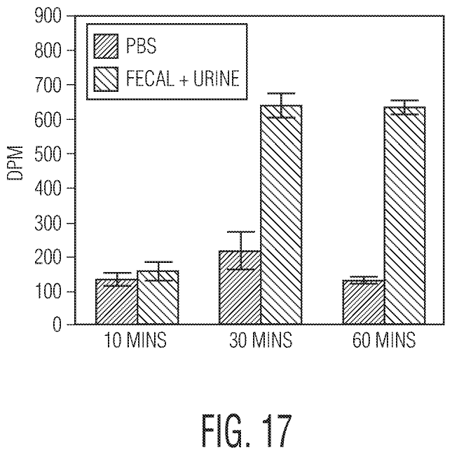

From this series of testing, it was evident that for the under-developed skin models with or without IL-6 the exposure time of one hour (i.e., 60 minutes) was too long when fecal enzymes and bile acids were present. To investigate the appropriate exposure time of urine simulant and fecal irritant mixture, the under-developed skin cultured in the standard medium condition exposure (skin model 10 treated with treatment solution 30 including IL-6 at 10 ng/mL after 3 days) with either PBS exposure as a control or a urine simulant and fecal irritant 1:1 mixture exposure was tested for ten, thirty, and sixty minutes. As illustrated in FIG. 17, a significant increase of skin capacitance was found for the thirty and sixty minute exposure times when comparing the control skin sample that was exposed to PBS to the skin sample that was exposed to the urine simulant and fecal irritant mixture.

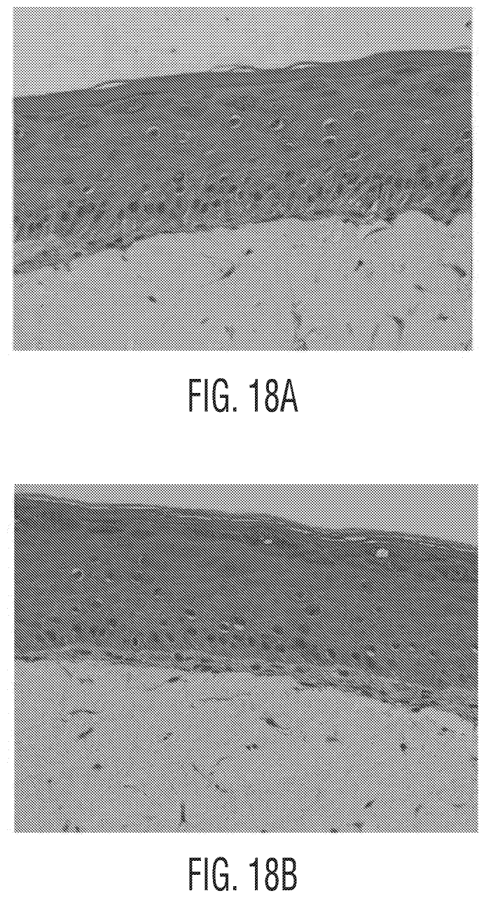

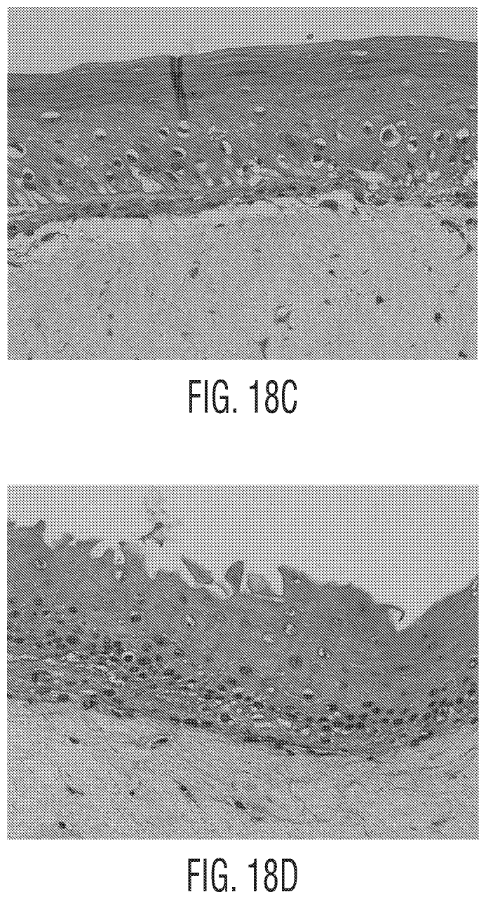

Histological images, illustrated in FIGS. 18A-18D, corresponded and verified the skin capacitance values. For example, FIG. 18A illustrates an under-developed skin model exposed to PBS (control) for ten minutes and shows a similar condition to the under-developed skin model exposed to the urine simulant and fecal irritant mixture for ten minutes illustrated in FIG. 18B. Exposure of the under-developed skin model to further lengths of time to PBS (such as 30 minutes and 60 minutes) did not result in any noticeable changes. However, FIG. 18C depicts an under-developed skin model exposed to the urine simulant and fecal irritant mixture for thirty minutes and sixty minutes, respectively, each of which demonstrate that the urine/fecal irritant mixture damaged epidermal cells after as early as after thirty minutes of exposure (FIG. 18C), and showed more apparent damage to epidermal cells after sixty minutes of exposure (FIG. 18D).

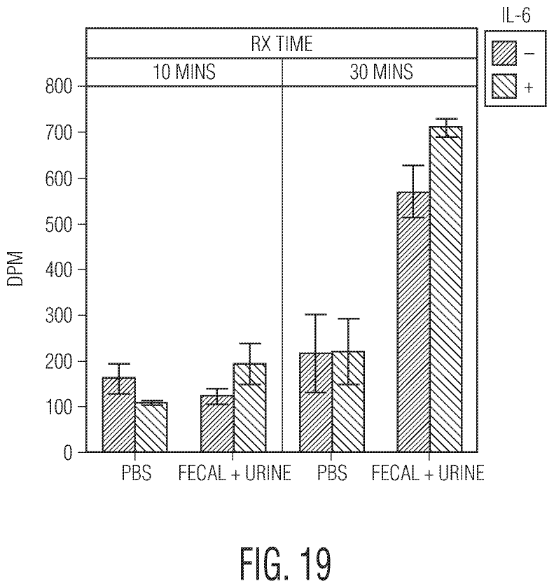

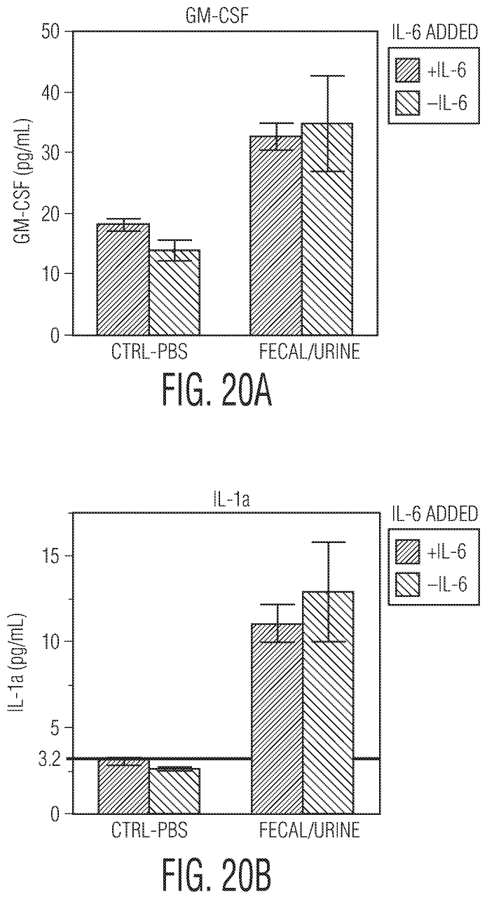

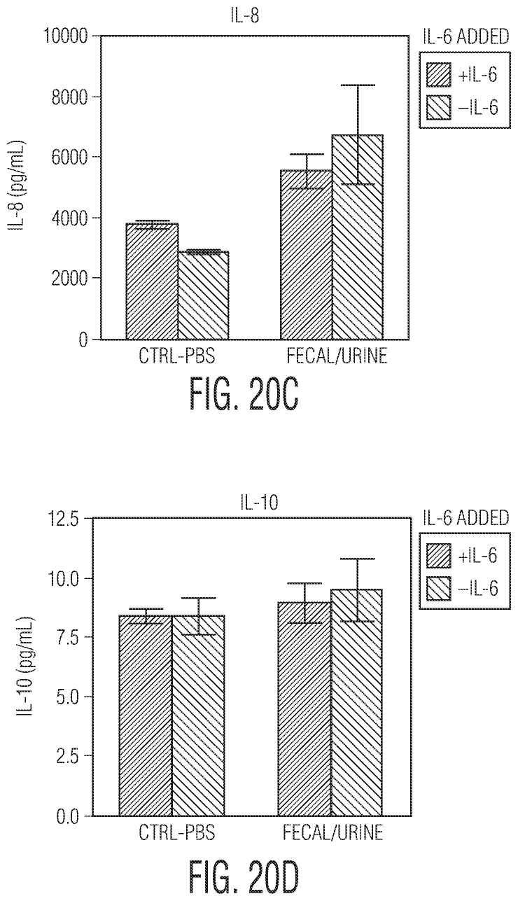

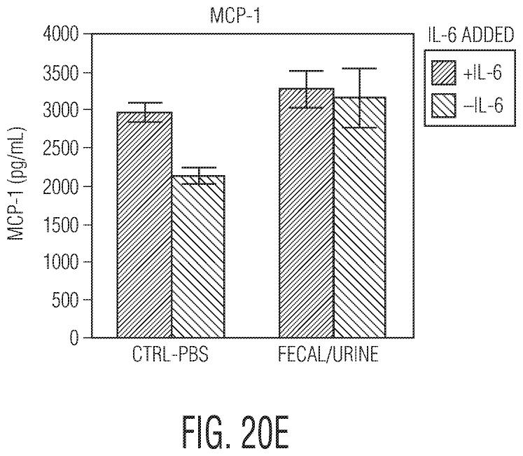

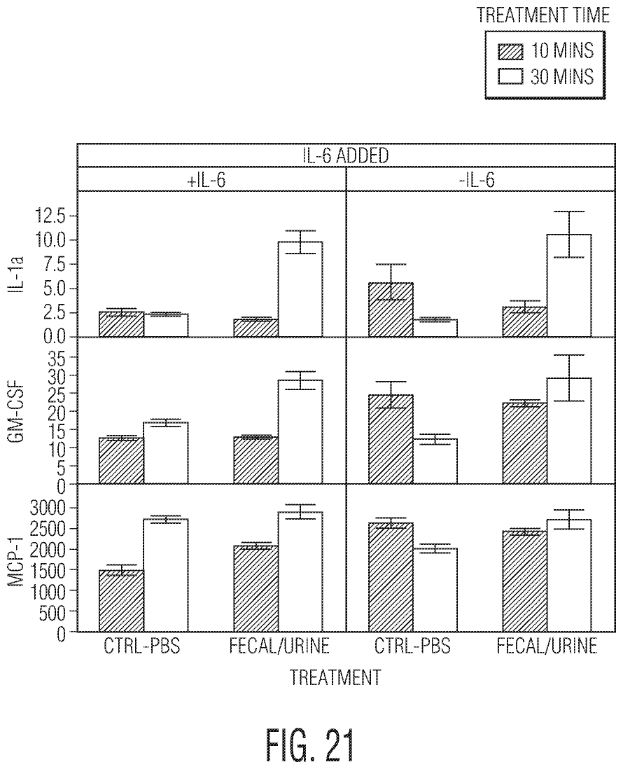

Subsequently, experiments were repeated twice at ten and thirty minute exposure times of the urine simulant/fecal irritant mixture for the under-developed skin model 10 with IL-6 treatment and the under-developed skin model without IL-6 treatment. As illustrated in FIG. 19, deterioration in skin barrier integrity as shown by the increase of skin capacitance was observed in the under-developed skin model that had no IL-6 treatment and that was exposed to urine/fecal mixture for 30 minutes, with a slight increase in the DPM values for the under-developed skin model 10 was treated with IL-6. FIGS. 20A-20E demonstrate that multiplex immunoassay testing showed that there was an up-regulation in cytokines of GM-CSF (FIG. 20A), IL-1a (FIG. 20B), and IL-8 (FIG. 20C) for the under-developed skin model 10 treated with IL-6 as well as the under-developed skin model not treated with IL-6 when exposed to the urine/fecal mixture after thirty minutes when compared to the control under-developed skin sample not treated with IL-6 and exposed to PBS. However, no such up-regulation of cytokines was exhibited for the under-developed skin models when only exposed to the urine/fecal mixture for ten minutes when compared to the same control, as shown in FIG. 21.

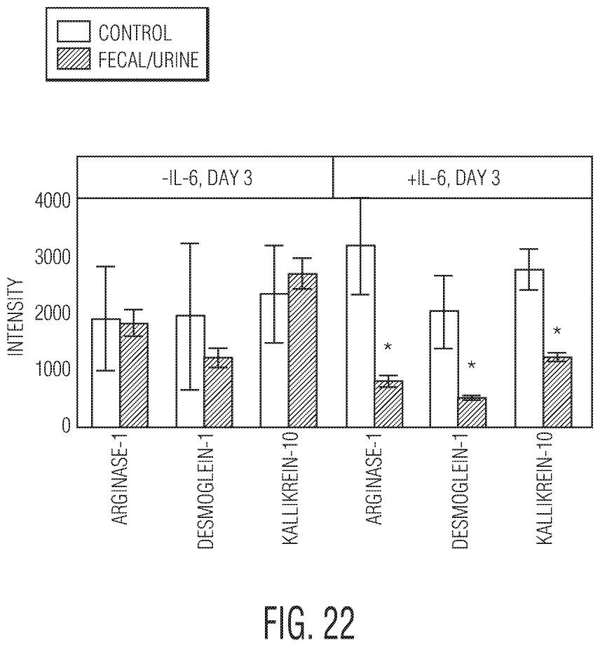

Although IL-6 treatment did not trigger further inflammatory cascades, an underdeveloped skin model 10 exposed to IL-6 was more susceptible to skin protein damage. For example, when challenged an under-developed skin model 10 exposed to IL-6 for 3 days to a mixture of urine simulant:fecal irritant (1:1) for 30 minutes, natural moisturizing factors (NM F) preprocess proteins, corneodemosomal proteins and serine proteases such as arginase-1, desmoglein 1 and kallikrein-10 were significantly decreased, as depicted in the right-hand side of the graph of FIG. 22. The same results cannot be replicated in an underdeveloped skin model not exposed to IL-6, as shown in the left-hand side of the graph of FIG. 22.

From the experimentation conducted, it was apparent that pre-developed skin cells exposed to 10 ng/mL to 100 ng/mL IL-6 resulted in the unusual acceleration of cell growth and aberrant epidermal development, which can be visualized by thicker epithelium and increased number of Ki67+proliferating cells in the basal and the suprabasal layer. The microarray gene profiling data further indicated that IL-6 may have effects on the metabolic process, signal transduction, and overall cell response to stress. While the changes in overall skin architecture cannot be distinguished in the H&E staining or in TEER readouts, IL-6 exposure causes deterioration of skin barrier proteins. For example, in terms of skin barrier proteins, IL-6 exposure during skin development decreased expression of proteins in S100 family were found in developing skin exposed to a treatment solution 10 including IL-6. S100 proteins play many crucial roles in calcium regulation and in skin barrier formation. Some of the proteins in S100 family, including S100A8 and/or S100A9 are also well known as antimicrobial peptides. The down-regulation of S100 proteins in skin models 10 exposed to treatment solutions 30 including IL-6 and the increased in proliferating cells beyond basal lamina suggested that IL-6 caused aberrant cell proliferation and delayed cell differentiation to form a proper barrier, creating an accurate model mimicking actual premature infant skin.

The urine/fecal exposure testing demonstrated that compared to fully-developed (mature) skin, which can tolerate up to 4 hours of urine/fecal irritant exposure, under-developed (premature) skin, regardless of whether the model was exposed to IL-6, can tolerate only up to thirty minutes of exposure of the same insult. Without addition of fecal irritants, fully-developed skin and under-developed skin can tolerate up to 24 hours of urine or PBS (wetness) insult without compromising skin morphology. Several proinflammatory cytokines, including GM-CSF, IL-1a, and IL-8 increased following urine and fecal irritant exposure.

Comparisons of the sensitivity of the under-developed skin model 10 exposed to IL-6 and the under-developed skin model not exposed to IL-6 each exposed to urine simulant/fecal irritant mixtures demonstrated that the skin model 10 exposed to IL-6 more accurately represented premature infant skin. While histological results did not indicate any significant morphological difference between these two skin models (those with and those without exposure to IL-6), biophysical measurements showed higher levels of skin barrier deterioration in IL-6 treated skin models 10. IL-8, GM-CSF, and IL-1a were increased compared to control for both the under-developed skin cultured with or without IL-6. MCP-1 and IL-10 levels did not change with fecal/urine irritant exposure in either of the under-developed tissue models.

By creating a skin model 10 that helps to more accurately replicate premature infant skin structure and behavior, the skin model 10 can provide a beneficial source for testing how various products interact or modify such skin without testing them on an actual user, serving as an important tool for research and development for a variety of products. Such testing can be used to screen new preventative regimens prior to larger multi-center clinical studies. Additionally, various testing with the skin model 10 can lead to further understanding of premature infant's skin. For example, studying of antimicrobial peptides and premature skin development can also be performed using the premature infant skin model 10.

### Procedures

### Preparation of Urine Simulant and Fecal Irritant

Urine simulant was prepared by combining 18.2 g/L urea, 7.5 g/L sodium chloride, 4.5 g/L potassium chloride, 4.8 g/L sodium phosphate 2 g/L creatinine, 50 mg/L. Final pH for the urine simulant was adjusted to 5.1, utilizing sodium hydroxide to increase pH or hydrochloric acid to decrease pH where necessary. Fecal irritant was prepared using 1500 μg/ml of a trypsin/chymotrypsin protease mixture (Specialty Enzymes and Biochemicals Co., Chino, Calif.) and a combination of bile acids. The bile acid mixture consisted of 6.5 mg/mL cholic acid sodium, 6.2 mg/mL deoxycholic acid sodium, and 3.1 mg/mL chenodeoxycholic acid sodium in phosphate buffered saline. Final pH was adjusted to 7.4, utilizing sodium hydroxide to increase pH or hydrochloric acid to decrease pH where necessary. All chemicals are available from Sigma-Aldrich (St. Louis, Mo.) unless otherwise noted.

### Measurement of Skin Model Resistance Using Transepithelial Electrical Resistance (TEER)

An Epithelial Volt-ohm meter 2 (EVOM2) available from World Precision Instruments, LLC (Sarasota, Fla.) with Endohm adaptor was used to measure the tight junction barrier integrity of the tested skin models. Unlike the Millicell® ERS-2 with chopstick-like electrodes, this instrument utilizes two fixed distance electrodes that minimize the error of measuring distance between readings. The total resistance (electrode-medium, membrane filter and the upper/lower compartment) was measured as ohm per square centimeter after applying an alternating current (AC) with low frequency (2.5 Hz) to the skin model. To use this EVOM2 instrument for measuring TEER of the skin models, the culture medium or treatment solution was replaced with 2.5 mL phosphate buffered saline (PBS), and 0.2 mL of additional PBS was applied on the skin model surface. Subsequently, cultured skin on the membrane insert was placed into the Endohm SnapWell chamber containing 6.0 mL PBS. The measurement was taken after 1 minute of equilibration time in the chamber. A high resistance reading represents good integrity of the epidermal tight junction (trans- and para-cellular pathways), whereas a low reading indicates the possibility of compromised or restructure of the tight junctions. As an example, a fully-developed skin model with excellent skin barrier typically has TEER values approximately about 1000 ohm/cm². Values below 100 ohm/cm² indicated severe skin barrier damage.

### Measurement of Skin Model Capacitance Using Phase Meter

A DPM 9003 instrument, available from NOVA Technology Corporation (Manchester, Mass.), was used to measure the surface electrical capacitance, which indirectly translated to cultured skin barrier integrity. The readings typically range between 90-800. A high DPM value indicates compromised skin barrier and low reading (below 200) indicates acceptable function of the skin barrier. Any value between 200-600 likely indicates partial (incomplete) barrier development. Good correlation between DPM readings, tissue morphology, (3-(4,5-Dimethylthiazol-2-yl)-2,5-Diphenyltetrazolium Bromide) (“MTT”) assays, and clinical graft take rate has been demonstrated previously, in vitro and in vivo. In order to perform a measurement in a six-well transmembrane plate, the original probe was modified to be smaller to fit within the well insert. This modified probe can be sterilized by gamma radiation or alcohol disinfection. To perform a routine reading, cultured skin within a six-well transmembrane insert was lay flat onto a petri dish. A DPM probe was gently placed on the skin and held steady for 5 seconds (dL5 mode).

### Measurement of IL-6 and IL-8 Generated from Skin Models

In-vitro SimpleStep ELISA® (Enzyme-Linked Immunosorbent Assay) kits model nos. ab178013 and ab174442, purchased from Abcam (Cambridge, Mass.), were used to measure baseline levels of IL-6 and interleukin-8 (“IL-8”), respectively, and their levels after urine insults. In brief, 50 μL volumes of culture supernatant collected 24 hours after controls or urine insults were diluted at ratios of 1:20 and 1:40 with a provided assay buffer (e.g., tris-buffered saline with low concentration of surfactant (SDS)), respectively, before addition into antibody-coated wells for either IL-6 or IL-8. After mixing with 50 μL of capture and detector antibodies for 60 minutes at 400 rpm, signal detection was commenced by the use of tetramethylbenzidine (“TMB”) substrate. Quantification of IL-6 or IL-8 level was performed using average 450 nm absorbance of supernatant samples compared to standard curves generated using manufacturer's protocol.

### Multiplex Immunoassay Testing

A customized multiplex immunoassay was used to monitor the level of secreted growth factors, cytokines and chemokines during skin maturation of the skin models and following the urine and fecal simulant insults of the skin models. The customized multiplex immunoassay used was a MILLIPLEX® MAP Human Cytokine/Chemokine Magnetic Bead Panel Immunology Multiplex Assay, Model No. HCYTOMAG-60K, available from Millipore (Bedford, Mass.), and included analytes of EGF, GM-CSF, IFNg, IL10, IL17A, IL-1a, IL1-b, IL-8, MCP-1, MIP-2a, MIP-1b, and TNF-a. Assays were performed according to the manufacturer's guidelines and fluorescent intensity was measured using a MAGPIX® multiplexing system available from Luminex Corporation (Austin, Tex.).

### Histology and Image Analysis

General tissue morphology was examined by H&E staining. Quarter pieces of skin samples were fixed overnight in 10% buffered formalin (Fisher Scientific, Pittsburg, Pa.) at 4° C. Subsequently, samples were transferred to PBS for shipment. Preparation of paraffin block, sectioning, H&E and Ki67 staining were performed by HSRL, Inc (Mount Jackson, Va.). Photomicrographs were captured using Nikon Eclipse upright microscope with NIS element software package and were at 20X magnification. The thickness of epidermis and stratum corneum thickness was analyzed using ImageJ. Measurements were done in triplicate on each tissue section. The number of Ki67+nuclei was quantitated using IHC Tool Box plugin using the settings for nuclei segmentation of window size: 25, seed size 1, final size 20; and settings for gland segmentation of Gaussian blur 2, open-by-recon. 40, and variance filter 5.

### Skin Barrier Proteins: Multiple Reaction Monitoring (MRM)

In order to establish a skin barrier protein panel to quantitatively measure 50-100 proteins simultaneously, LC-MS/MS shotgun proteomics of the human skin tissues and the fully-developed skin models derived from three different donors was used for protein inventory establishment. To do so, 0.5 cm² skin samples were lysed and homogenized with stainless beads in tissue lyser beadmills. After determination of protein concentration, lysed samples were subjected to denaturation, reduction/alkylation of cysteine, trypsin digestion, desalting and clean up with C18 spin columns. Determination of protein concentration and peptide concentrations were carried out using Pierce BCA Protein Assay Kit, available from ThermoFisher Scientific, Inc. (Waltham, Mass.) and SPECTROstar® Nano UV/VIS spectrometer available from BMG Labtech GmbH (Ortenberg, Germany). MS data was searched with MaxQuant against UNIPROT human protein database allowing for two missed cleavages and variable modification (N-terminal acetylation and methionine oxidation).

Results from protein inventory in skin samples were used for selection of relevant proteins that represent the state (healthy/unhealthy) of the skin barrier. Selected set of barrier proteins were verified in GO biological process and molecular functions using BINGO app plug-in within Cytoscape 3.0.

### Microarrav RNA Testing

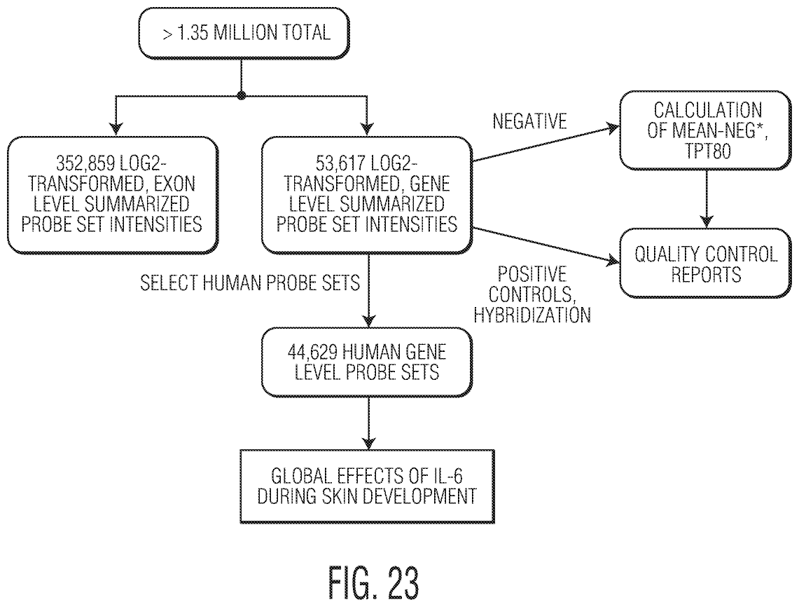

In order to simultaneously investigate the changes of human genes, total RNA was prepared from 0.25 cm² of the in vitro premature infant skin models exposed to the IL-6 treatment solution, as well as the comparative in vitro premature infant skin models discussed herein. Yield and quality of total RNA were verified using agarose gel stained with ethidium bromide. Biotinylated cRNA were hybridized onto Affymetrix GeneChip® Human Gene ST 2.0 arrays according to manufacturer's instructions. Data analysis was performed using online freeware according to the schematic diagram illustrated in FIG. 23.

Robust Multi-array Average (“RMA”) was performed on raw data values in that raw intensity values are background corrected, loge transformed and then quantile normalized. Next, a linear model is fit to the normalized data to obtain an expression measure for each probe set on each array. The RMA transformed data were normalized using quantile and probe set summarization (gene level/exon level). Quality controls were carried out by the use of on chip hybridization controls, housekeeping controls as well as principle component analysis. NIA Array analysis software (http://lgsun.grc.nia.nih.gov/ANOVA/) was used to perform ANOVA, Tukey t-test (p<0.05) and false discovery rate calculation. For data analytic strategies, candidate genes were selected based on the global effects of IL-6 during skin development, having controls and IL-6 treated under-developed skin models taken on days 1, 3, and 7.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present disclosure should be assessed as that of the appended claims and any equivalents thereto.

### Embodiments

Embodiment 1: A method of creating a premature infant skin model, the method comprising: providing neonatal skin cells; exposing the neonatal skin cells to a treatment solution comprising interleukin-6 for a treatment period; and removing the treatment solution from the neonatal skin cells after the treatment period to provide the three-dimensional premature infant skin model.

Embodiment 2: The method of embodiment 1, wherein the neonatal skin cells comprise neonatal dermal skin cells and neonatal epidermal skin cells.

Embodiment 3: The method of embodiment 1 or 2, wherein exposing the neonatal skin cells to a treatment solution comprising interleukin-6 comprises culturing the neonatal skin cells at an air-liquid interface.

Embodiment 4: The method of any one of the preceding embodiments, wherein the treatment period is less than seven days.

Embodiment 5: The method of any one of the preceding embodiments, wherein the treatment period is greater than one day.

Embodiment 6: The method of any one of embodiments 1-3, wherein the treatment period is between two to six days.

Embodiment 7: The method of any one of the preceding embodiments, wherein the interleukin-6 is provided at a concentration of from about 10 ng/mL to about 100 ng/mL by total weight/volume of the treatment solution.

Embodiment 8: The method of any one of the preceding embodiments, wherein the treatment solution further comprises a base medium of Dulbecco's Modified Eagles Medium and at least one growth factor.

Embodiment 9: The method of embodiment 2, further comprising: culturing the neonatal dermal skin cells in a culture medium for an initial culture period; and adding the neonatal epidermal skin cells on top of the neonatal dermal skin cells after the initial culture period for a secondary culture period.

Embodiment 10: The method of embodiment 9, wherein the initial culture period is from four to seven days.

Embodiment 11: The method of embodiment 9 or 10, wherein the secondary culture period is at least two days.

Embodiment 12: The method of any one of the preceding embodiments, wherein the neonatal skin cells are from a skin equivalent.

Embodiment 13: A premature infant skin model prepared according to the method of any one of the preceding embodiments.

Embodiment 14: A method of creating a three-dimensional premature infant skin model, the method comprising: providing neonatal skin cells, the neonatal skin cells comprising neonatal dermal skin cells and neonatal epidermal skin cells; exposing the neonatal skin cells to a treatment solution comprising interleukin-6 for a treatment period, the interleukin-6 being provided at a concentration of from about 10 ng/mL to about 100 ng/mL by total weight/volume of the treatment solution; and removing the treatment solution from the neonatal skin cells after the treatment period to provide the three-dimensional premature infant skin model.

Embodiment 15: The method of embodiment 14, wherein the treatment period is between one and seven days.

Embodiment 16: A three-dimensional premature infant skin model prepared according to the method of embodiment 14 or 15.

What is claimed is:

1. A method of creating a premature infant skin model, the method comprising:
providing neonatal skin cells;
exposing the neonatal skin cells to a treatment solution comprising interleukin-6 for a treatment period; and
removing the treatment solution from the neonatal skin cells after the treatment period to provide the three-dimensional premature infant skin model.

2. The method of claim 1, wherein the neonatal skin cells comprise neonatal dermal skin cells and neonatal epidermal skin cells.

3. The method of claim 1, wherein exposing the neonatal skin cells to a treatment solution comprising interleukin-6 comprises culturing the neonatal skin cells at an air-liquid interface.

4. The method of claim 1, wherein the treatment period is less than seven days.

5. The method of claim 1, wherein the treatment period is greater than one day.

6. The method of claim 1, wherein the treatment period is between two to six days.

7. The method of claim 1, wherein the interleukin-6 is provided at a concentration of from about 10 ng/mL to about 100 ng/mL by total weight/volume of the treatment solution.

8. The method of claim 1, wherein the treatment solution further comprises a base medium of Dulbecco's Modified Eagles Medium and at least one growth factor.

9. The method of claim 2, further comprising:
culturing the neonatal dermal skin cells in a culture medium for an initial culture period; and
adding the neonatal epidermal skin cells on top of the neonatal dermal skin cells after the initial culture period for a secondary culture period.

10. The method of claim 9, wherein the initial culture period is from four to seven days.

11. The method of claim 9, wherein the secondary culture period is at least two days.

12. The method of claim 2, wherein the neonatal skin cells are from a skin equivalent.

13. A premature infant skin model prepared according to the method of claim 1.

14. A method of creating a three-dimensional premature infant skin model, the method comprising:
providing neonatal skin cells, the neonatal skin cells comprising neonatal dermal skin cells and neonatal epidermal skin cells;
exposing the neonatal skin cells to a treatment solution comprising interleukin-6 for a treatment period, the interleukin-6 being provided at a concentration of from about 10 ng/mL to about 100 ng/mL by total weight/volume of the treatment solution; and
removing the treatment solution from the neonatal skin cells after the treatment period to provide the three-dimensional premature infant skin model.

15. The method of claim 14, wherein the treatment period is between one and seven days.

16. A three-dimensional premature infant skin model prepared according to the method of claim 14.
